(19) United States
(12) Patent Application Publication
(10) Pub. No.: US 2025/0064874 A1
(43) Pub. Date: Feb. 27, 2025

(54) Mushroom Spore, Cannabinoid, and Specified Compositions

(71) Applicant: Maria Eliza Valentino, Riegelsville, PA (US)

(72) Inventor: Maria Eliza Valentino, Riegelsville, PA (US)

(21) Appl. No.: 18/812,547

(22) Filed: Aug. 22, 2024

Related U.S. Application Data
(60) Provisional application No. 63/578,224, filed on Aug. 23, 2023.

(51) Int. Cl. A61K 36/074; A61K 31/00; A61K 31/122; A61K 31/198; A61K 31/355; A61K 31/375; A61K 31/405; A61K 31/4172; A61K 31/593; A61K 36/062; A61K 36/068; A61K 36/07; A61K 36/076; A61K 36/82; A61K 47/20; A61P 17/06; A61P 17/10; A61P 25/20; A61P 25/24; A61P 25/28; A61P 29/00; A61P 35/00

(52) U.S. Cl. CPC A61K 36/074; A61K 31/122; A61K 31/198; A61K 31/355; A61K 31/375; A61K 31/405; A61K 31/4172; A61K 31/593; A61K 31/658; A61K 36/062; A61K 36/068; A61K 36/07; A61K 36/071; A61K 36/076; A61K 36/078; A61K 36/742; A61K 36/82; A61K 47/20; A61P 17/06; A61P 17/10; A61P 25/20; A61P 25/24; A61P 25/28; A61P 29/00; A61P 35/00

(57) ABSTRACT

A composition for upregulating the immune system, endocannabinoid system and main bodily systems while promoting mental stability for optimal wellness. Also provided is a method for enhancing/stimulating the immune, endocannabinoid and/or main bodily systems of a patient, particularly a mammal with a disease or disorder, which includes a step of administering the composition to the patient. The composition includes a combination of Mushroom Spore Oil and/or Spore Powder and at and at least one of b1) a cannabinoid, b2) DMSO/Dimethyl Sulfoxide, b3) minerals, b4) botanicals, b5) Vitamins, b6) Amino Acids, and b7) fungi.

This nonprovisional utility application claims priority to provisional application No. 63/578,224, filed Aug. 23, 2023, the entire contents of which is incorporated herein by reference in its entirety.

### TECHNICAL FIELD

The disclosure relates to a composition comprising: a) a mushroom spore oil and/or powder; and at least one of b1) a cannabinoid, b2) DMSO/Dimethyl Sulfoxide, b3) minerals, b4) botanicals, b5) Vitamins, b6) Amino Acids, and b7) fungi, and its use in a method for enhancing/stimulating the immune, endocannabinoid and/or main bodily systems of a patient, particularly a mammal with a disease or disorder.

### BACKGROUND

Ganoderma (Ganoderma lucidum Leyss ex Fr. Karst, a.k.a. “Reishi”) is a polyporous fungus. It belongs to the class Basidiomycetes, the family Polypolaceae, and the genus Ganoderma. Since ancient times, ganoderma has been praised as a miracle fungus for its capability of prolonging human life. It is believed that the medicinal effects of ganoderma lie upon the natural or bioactive substances it produces which can stimulate or modulate the neuro-endocrino-immuno system of a mammal to fight off diseases. Ganoderma is also well known for its antitumor and immune enhancing properties, (Kim et al., Int. J. Mol. Med. (1999), 4(3):273-277), cardiovascular effects (Lee et al., Chem. Pharm. Bull. (1990), 38:1359-1364), as well as free radical scavenging and antihepatotoxic activities (Lin et al., J. Ethnopharmacol., (1995), 47(1):33-41).

Ganoderma is the most rare and valuable herb in Chinese medicine. It is known in China for over 5,000 years as “ling zhi”. There are a variety of ganoderma, for instance, G. lucidum (red), G. applanatum (brown), G. tsugae (red), G. sinense (black), and G. oregonense (dark brown). However, due to the fact that wild types of ganoderma only grow naturally and very rarely on aged trees in steep mountains, research which requires a constant supply of high quantity and quality of ganoderma has rarely been conducted.

Although it is believed that the spores of ganoderma represent the essence of ganoderma because they contain all the bioactive substances of ganoderma, most of the ganoderma studies are conducted using the fruit body or mycelium of ganoderma as experimental materials. Ganoderma spores are rarely studied.

Ganoderma spores are tiny, mist-like spores of about 5 μm to 8 μm in size which have extremely hard and resilient, double-layer epispores, thus making them difficult to break open. The ganoderma spores normally scatter at the pelius of mature ganoderma. When mature, the ganoderma spores are ejected from the pileus. Such ejected ganoderma spores are collectively called “spore powders”. In the wild, the “spore powders” are difficult to collect because: (1) the germination rate (i e., about 3-15%) of the spores is extremely low; (2) the ejection period is relatively short (i.e., approximately 10 days per lifecycle); and (3) some environmental factors, such as wind and rain, may also hinder the collection of the spores. In addition, the substances contained in the collected spores are difficult to extract due to the resiliency of the epispores.

Cannabinoids are a group of chemicals known to activate cannabinoid receptors in cells. These chemicals, which are found in cannabis plants, are also produced from endogenous form in humans and other animals, which are called endocannabinoids. Synthetic cannabinoids are chemicals with structures similar to cannabinoids or endocannabinoids.

Plant cannabinoids can also be isolated so that they are “essentially pure” compounds. These isolated cannabinoids are essentially free of other natural compounds, such as other minor cannabinoids and molecules such as terpenes.

The essentially pure compounds have a degree of purity of at least 95% of the total weight. It has been suggested that some essentially pure cannabinoids (either synthetic or isolated) are neuroprotective agents, either by direct antagonism of the NMDA receptor or by reducing the influx of calcium ions into the cell by other means, such as binding to cannabinoid receptors.

It was found that the toxicity of glutamate could be prevented to some extent by isolated or synthetic or cannabidiol (CBD), (Hampson et al., 1998). The cannabinoids were tested in vitro in neuronal cultures exposed to glutamate. However, further investigations of an in vivo study conducted by the same group failed to find a difference between animals treated with isolated or synthetic CBD and animals treated with placebo (Rosenthal et al., 2000).

The treatment of diseases including epilepsy, bradycardia, tachycardia, hypertension, hypotension, inflammation, multiple sclerosis (MS), spinal cord injury, neuropathies, inflammatory bowel disease (IBD), stroke, head injury and immune diseases using cannabis extracts is described in US 2004/034108, GB2392093, WO 2004/016246, WO 02/064109 and WO 03/037306.

WO 03/105800 describes the use of cannabis extracts to treat MS paralysis, movement disorders, asthma, epilepsy, withdrawal symptoms of alcoholism, Parkinson's disease, Alzheimer's disease and arthritis. WO 02/069993 describes the use of cannabis extracts as antidepressants, anxiolytics, antiepileptics and as an anti-inflammatory.

### SUMMARY

In an aspect, the present invention relates to a composition for upregulating the immune system, upregulating the endocannabinoid system, upregulating the main bodily systems and improving mental stability for optimal wellness. Embodiments of the invention are described in the following numbered sentences.
1. The composition comprises: a) a mushroom spore oil and/or powder; and at least one of b1) a cannabinoid, b2) DMSO/Dimethyl Sulfoxide, b3) minerals, b4) botanicals, b5) Vitamins, b6) Amino Acids, and b7) fungi; wherein the mushroom spore oil and/or powder can be based on at least one of the following mushrooms:
   i. Reishi (Ganoderma lucidum)
   ii. Lion's Mane (Hericium Erinaceus),
   iii. Cordyceps (Cordyceps Militaris),
   iv. Turkey Tall (Trametes versicolor/Coriolus versicolor),
   v. Agaricus (Agaricus Blazei),
   vi. Chaga (Inonotus Obliquus),
   vii. Tremella (Tremella fuciformis),
   viii. Shiltake (Lentinula edodes),
   ix. Maltake (Grifola frondosa),
   x. Oyster (Pleurotus ostreatus),
   xi. Indian Oyster (Pleurotus Pulmonarius)
   xii. Porta (Wolfipora cocos),
   xiii. Boletus (Boletus edulis),
   xiv. Morel (Morchella esculenta),
   xv. Amanita (Amanita Muscaria),
   xvi. Trumpet (Pleurotus eryngii),
   xvii. Psilocybe,
   xviii. Usnea Lichen,
   xix. Lobaria pumponaria Lichen,
   xx. Cetraria Islandica/Icelandic Moss Lichen, and
   xxi. Evernia Lichen.
2. The composition according to sentence 1, further comprising both coffee and/or specified botanicals and a cannabinoid.
3. The composition of any one of the previous sentences comprising Reishi (Ganoderma lucidum) spore oil and/or powder.
4. The composition of any one of the previous sentences, comprising a mushroom fruit body and/or mycelium extract of at least one of components i-xxi.
5. The composition of any one of the previous sentences, further comprising b1) a cannabinoid.
6. The composition of any one of the previous sentences, wherein the cannabinoid is including but not limited to a full spectrum cannabinoid, broad spectrum cannabinoid, isolated cannabinoid, cannabidiol (CBD), Hemp CBD, tetra-hydrocannabinol (THC), cannabigerol (CBG), cannabinol (CBN), cannabichromene (CBC) or mixtures thereof.
7. The composition of any one of the previous sentences, wherein the cannabinoid is a full spectrum cannabinoid, broad spectrum cannabinoid, and/or isolated cannabinoid.
8. The composition of any one of the previous sentences, comprising cannabis.
9. The composition of any one of the previous sentences, comprising at least one terpene.
10. The composition of any one of the previous sentences, comprising at least one terpene selected from the group consisting of Limonene, Caryophyllene, Humulene, Pinene, Myrcene, Bisabolol, Carene, Terpinene, Borneol, Camphene, Eucalyptol, Geraniol, Phytol, Terpinolene, Nerolidol, and Valencene.
11. The composition of any one of the previous sentences, comprising DMSO.
12. The composition of any one of the previous sentences, comprising at least one mineral.
13. The composition of any one of the previous sentences, comprising at least one mineral selected from the group consisting of magnesium, potassium, silica, sodium, zinc, boron, chloride, iron, Himalayan salt, fluoride, and shilajit.
14. The composition of any one of the previous sentences, comprising magnesium sulfate.
15. The composition of any one of the previous sentences, comprising a topical formulation prepared by combining 1-100 gallons of water at 32-40° C., 100-700 grams of Magnesium sulfate and 500-1500 mg of Reishi Mushroom Spore Powder; or a topical formulation prepared by combining 10-40 gallons of water at 32-40° C., 300-600 grams of Magnesium sulfate and 900-1200 mg of Reishi Mushroom Spore Powder.
16. The composition of any one of the previous sentences, comprising at least one botanical.
17. The composition of any one of the previous sentences, comprising at least one botanical selected from the group consisting of Hemp, Coffee, Tea, Green Tea, Black tea, Matcha, Chai, Cacao, Cloves, Baobab, Ashwaganda, Ginseng, Moringa, Brahmi/Bacopa Monieri, Hibiscus, He Shoo Woo, Pau D'Arco, Matcha, Irish Sea Moss, Cinnamon, Oregano, Neem, Nettles, Arnica, Calendula, Comfrey, Chamomile, Valerian Root, Bladderwrack, Burdock, Tamanu, turmeric, Horsetail, Saw palmetto, Ginger, Turmeric, Cacao, Rooibos, Fennel, Echinacea, Basil, Thyme, Lemongrass, Lemon balm, Passionflower, Guanabana, Tulsi, Fo-ti, Vanilla, Coconut, Cedarwood, Jasmine, Imortelle, Lavender, Rose, Geranium, Eucalyptus, Peppermint, Spearmint, Rosemary, Ylang Ylang, Lemon, Citronella, Sage, Plumeria, Imortelle, Shisandra, Astraxanthan (Algea derived), Maca, Egyptian Blue Lotus, boga, Kratom, coconut, Almond, Sweet Almond, Olive, Jojoba, Argan, Avocado, Grape/Grapeseed, Castor, Prickly Pear, Sea Buckthorn, Apricot, blackseed, evening primrose, rosehip, flax, Carrot, sunflower, Cocoa, Mango, Shea, Lucuma, Indian rhubarb root (Rheum palmatum), Sheep sorrel (Rumex acetosella), Slippery elm (Ulmus rubra), Dandelion, Apple (Apple Pectin), Pineapple (Bromelain Pineapple Enzyme), Spirulina, Chlorella, Milk thistle, Wormwood, Black walnut, Shilajit, and Pine.
18. The composition of any one of the previous sentences, comprising coffee.
19. The composition of any one of the previous sentences, comprising ground coffee.
20. The composition of any one of the previous sentences, comprising brewed coffee.
21. The composition of any one of the previous sentences, comprising tea and cannabinoid.
22. The composition of any one of the previous sentences, comprising hemp cannabinoid.
23. The composition of any one of the previous sentences, comprising full spectrum cannabinoid and coffee.
24. The composition of any one of the previous sentences, comprising Reishi Spore Powder, ashwaganda and coffee.
25. The composition of any one of the previous sentences, comprising at least one vitamin.
26. The composition of any one of the previous sentences, comprising at least one vitamin selected from the group consisting of Vitamin E, Vitamin C, Vitamin D, and Vitamin K.
27. The composition of any one of the previous sentences, comprising at least one amino acid.
28. The composition of any one of the previous sentences, comprising at least one amino acid selected from the group consisting of Leucine, Valine, Isoleucine, Phenylalanine, Lysine, Histidine, Methionine, Tryptophan, and Threonine.
29. The composition of any one of the previous sentences, comprising at least one mushroom fruiting body and/or mycelium.
30. The composition of any one of the previous sentences, comprising at least one mushroom fruiting body and/or mycelium selected from the group consisting of Reishi (Ganoderma lucidum), Lion's Mane (Hericium Erinaceus), Cordyceps (Cordyceps Militaris), Turkey Tail (Trametes versicolor/Coriolus versicolor), Agaricus (Agaricus Blazei), Chaga (Inonotus Obliquus), Tremella (Tremella fuciformis), Shiitake (Lentinula edodes), Maitake (Grifola frondosa), Oyster (Pleurotus ostreatus), Indian Oyster (Pleurotus Pulmonarius), Poria (Wolfiporia cocos), Boletus (Boletus edulis), Morel (Morchella esculenta), Amanita (Amanita Muscaria), Trumpet (Pleurotus eryngii), and Psilocybe.
31. The composition of any one of the previous sentences, comprising Reishi Spore Powder and/or oil and Lion's Mane fruiting body powder.
32. The composition of any one of the previous sentences, which is formulated to be administered by a mode selected from the group consisting of intravenous injection, intramuscular injection, subcutaneous injection, topical administration, transdermal patch, suppository and oral.
33. The composition of any one of the previous sentences, which is formulated to be administered orally, and is in the form of a liquid, powder, a tablet or capsule.
34. The composition of any one of the previous sentences, which is formulated for topical administration as a solution, gel, paste, ointment, cream, jelly, spay or suspension.
35. The composition of any one of the previous sentences, which does not include a cannabinoid or cannabis.
36. A kit comprising the composition of any one of the previous sentences comprising: a) a composition formulated for oral administration; and b) a composition formulated for topical administration.
37. A kit comprising the composition of any one of sentences 1-35, comprising: a) a composition formulated for oral administration; b) a composition formulated for topical administration; and c) a composition formulated for administration as a suppository.
38. A method of upregulating the immune system comprising administering a therapeutically effective amount of the composition of any one of sentences 1-35, to a patient in need thereof.
39. A method of upregulating the endocannabinoid system comprising administering a therapeutically effective amount of the composition of any one of sentences 1-35, to a patient in need thereof.
40. A method of upregulating the main bodily systems comprising administering a therapeutically effective amount of the composition of any one of sentences 1-35, to a patient in need thereof.
41. A method of improving mental stability comprising administering a therapeutically effective amount of the composition of any one of sentences 1-35, to a patient in need thereof.
42. A method of upregulating the immune system, upregulating the endocannabinoid system, upregulating the main bodily systems and improving mental stability comprising administering a therapeutically effective amount of the composition of any one of sentences 1-35, to a patient in need thereof.
43. A method of reducing symptoms of at least one of obesity, insomnia, depression, dementia, brain fog, vision loss, anxiety, obsessive compulsive disorder, cancer, lupus, nausea, rheumatoid arthritis, migraines, poly cystic ovarian syndrome, menstrual pain, menopause, diabetes, nerve damage, sciatica, Epstein Barr virus, hypothyroidism, post-traumatic stress disorder, herpes, shingles, tremors, hpv, pathogenic viruses, asthma, Crohn's disease, Parkinson's disease, diverticulitis, osteoporosis, carpal tunnel syndrome, allergies, high cholesterol, inflammation, eczema, psoriasis, acne, tooth decay, oral bacteria, hair loss, rosacea, dermatitis, shingles, fungal infection, memory loss, trichotillomania, dermatillomania, urinary tract infections, wound, cancer and the effects of cancer-related treatments such as cachexia and wasting, damage caused by concussion and related symptoms, and symptoms caused from breast implant illness comprising administering a therapeutically effective amount of the composition of any one of sentences 1-35, to a patient in need thereof.
44. A method of reducing symptoms of psoriasis, acne, or rosacea, comprising administering a therapeutically effective amount of the composition of any one of sentences 1-35, to a patient in need thereof.
45. A method of reducing pain, reducing inflammation or improving cell rejuvenation, comprising administering a therapeutically effective amount of the composition of any one of sentences 1-35, to a patient in need thereof.
46. A method of reducing symptoms of depression, anxiety or hypothyroidism, comprising administering a therapeutically effective amount of the composition of any one of sentences 1-35, to a patient in need thereof.
47. A method of reducing symptoms of psoriasis, acne, and rosacea, comprising administering a therapeutically effective amount of the composition of any one of sentences 1-35, to a patient in need thereof.
48. A method of reducing pain, reducing inflammation and improving cell rejuvenation, comprising administering a therapeutically effective amount of the composition of any one of sentences 1-35, to a patient in need thereof.
49. A method of reducing symptoms of anxiety and hypothyroidism, comprising administering a therapeutically effective amount of the composition of any one of sentences 1-35, to a patient in need thereof.
50. A method of reducing symptoms of depression, comprising administering the composition according to any one of sentences 1-35, to a patient in need thereof.
51. A method of reducing symptoms of depression, comprising administering chamomile tea, 100-700 mg of reishi mushroom spore powder and/or oil and 1-25 mg of Full Spectrum CBD to a patient in need thereof; or comprising administering chamomile tea, 200-600 mg of reishi mushroom spore powder and/or oil and 10-20 mg of Full Spectrum CBD to a patient in need thereof.
52. A method of reducing symptoms of acne, comprising administering the composition according to any one of sentences 1-35, to a patient in need thereof.
53. A method of reducing symptoms of acne, comprising administering a topical composition comprising hemp cannabinoid and reishi mushroom spore powder and/or oil to the effected skin of a patient in need thereof.
54. A method of reducing symptoms of at least two of rosacea, eczema, psoriasis, stretch marks and acne, comprising administering the composition according to sentences 1-35, to a patient in need thereof.
55. A method of reducing symptoms of at least two of rosacea, eczema, psoriasis, stretch marks and acne comprising administering a topical composition comprising ground roasted coffee, 100-700 mg mushroom spore oil, and 1-25 mg full spectrum CBD to the effected skin of a patient in need thereof; or comprising administering a topical composition comprising ground roasted coffee, 100-700 mg mushroom spore oil, and 1-25 mg full spectrum CBD to the effected skin of a patient in need thereof.
56. A method of reducing symptoms of at least two of brain fog, indigestion, jitters, anxiety, depression, and sleeplessness, comprising administering the composition according to any one of sentences 1-35, to a patient in need thereof.
57. A method of reducing symptoms of at least two of brain fog, indigestion, jitters, anxiety, depression, and sleeplessness comprising administering coffee, mushroom spore powder and/or oil, and Ashwaganda to a patient in need thereof; or comprising administering an oral composition of 100-1000 mL of brewed coffee, 100-1000 mg mushroom spore powder and/or oil, and 0.1-10 g of Ashwaganda to a patient in need thereof; or comprising administering an oral composition of 150-500 mL of brewed coffee, 300-900 mg mushroom spore powder and/or oil, and 1-6 g of Ashwaganda to a patient in need thereof.
58. A method of reducing symptoms of at least two of brain fog, upset stomach, and lack of energy, comprising administering the composition according to any one of sentences 1-35, to a patient in need thereof.
59. A method of reducing symptoms of at least two of brain fog, upset stomach, and lack of energy, comprising administering Reishi Spore Powder and/or oil with Lion's Mane fruiting body powder to a patient in need thereof.
60. The composition of any one of sentences 1-35, or method of any one of sentences 38-59, wherein the oral dosage level is 0.1 mg-20,000 mg Ganoderma Lucidum spore oil and/or spore powder per day, and 0.1-10,000 mg of the one or more cannabinoids per day; or the oral dosage level is 0.1-5000 mg Ganoderma Lucidum spore oil per day, and 0.1-10000 mg of the one or more cannabinoids per day; or the oral dosage level is 90-3200 mg Ganoderma Lucidum spore oil per day, and 10-250 mg of the one or more cannabinoids per day.
61. The composition of any one of sentences 1-35, or method of any one of sentences 38-59, wherein the transdermal dosage level is 1-200 mg Ganoderma Lucidum spore oil/powder per day, and 1-100 mg of the one or more cannabinoids per day; the transdermal dosage level is 5-100 mg Ganoderma Lucidum spore oil per day, and 1-50 mg of the one or more cannabinoids per day; or the transdermal dosage level is 10-75 mg Ganoderma Lucidum spore oil per day, and 1-20 mg of the one or more cannabinoids per day.
62. The composition of any one of sentences 1-35, or method of any one of sentences 38-59, wherein the composition is in a suppository form, and the suppository dosage level is 1-1000 mg Ganoderma Lucidum spore oil per day, and 1-1000 mg of the one or more cannabinoids per day; or the suppository dosage level is 25-500 mg Ganoderma Lucidum spore oil per day, and 30-300 mg of the one or more cannabinoids per day; or the suppository dosage level is 75-200 mg Ganoderma Lucidum spore oil per day, and 50-200 mg of the one or more cannabinoids per day.

### DETAILED DESCRIPTION

In an aspect of the disclosure is a composition comprising: a) a mushroom spore oil and/or powder; and at least one of b1) a cannabinoid, b2) DMSO/Dimethyl Sulfoxide, b3) minerals, b4) botanicals, b5) Vitamins, b6) Amino Acids, and b7) fungi. In another aspect of the disclosure is a method for enhancing/stimulating the immune, endocannabinoid and/or main bodily systems of a patient, particularly a mammal with a disease or disorder comprising administering said composition to said patient.

In an embodiment, the mushroom spore oil and/or powder is based at least one of the following mushrooms:
i. Reishi (Ganoderma lucidum)
ii. Lion's Mane (Hericium Erinaceus),
iii. Cordyceps (Cordyceps Militaris),
iv. Turkey Tail (Trametes versicolor/Coriolus versicolor),
v. Agaricus (Agaricus Blazei),
vi. Chaga (Inonotus Obliquus),
vii. Tremella (Tremella fuciformis),
viii. Shiitake (Lentinula edodes),
ix. Maitake (Grifola frondosa),
x. Oyster (Pleurotus ostreatus),
xi. Indian Oyster (Pleurotus Pulmonarius).
xii. Poria (Wolfiporia cocos),
xiii. Boletus (Boletus edulis),
xiv. Morel (Morchella esculenta),
xv. Amanita (Amanita Muscaria),
xvi. Trumpet (Pleurotus eryngii),
xvii. Psilocybe,
xviii. Usnea Lichen,
xix. Lobaria pumponaria Lichen,
xx. Cetraria Islandica/Icelandic Moss Lichen, and
xxi. Evernia Lichen.

The composition can comprise the mushroom fruit body and/or mycelium extract of at least one of components i)-xxi).

The Ganoderma Lucidum (Reishi) spore oil employed in the composition of the present invention is the oil and powder extracted from the shells of Ganoderma Lucidum spores. The traditional extracting method of Ganoderma spore oil generally adopts methyl alcohol or ethanol. A more efficient method for extracting Ganoderma Lucidum spores oil is by using a subcritical mixed solvent. Another method involves using supercritical CO₂ extraction techniques. The process involves cleansing Ganoderma Lucidum spores; breaking the walls of the Ganoderma Lucidum spores to obtain Ganoderma Lucidum spore powder; preparing the wall-broken Ganoderma Lucidum spore powder into particles through a granulator by adopting a dry pressing method; putting the particles into a supercritical CO₂ extraction kettle for extraction; isolating coarse Ganoderma Lucidum spore oil; rectifying and separating to obtain the Ganoderma Lucidum spore oil.

Ganoderma Lucidum spore oil may contain one or more of the following components: polysaccharides, triterpenes, beta-glucans and adaptogens.

The at least one cannabinoid is preferably obtained from one or both of the cannabis plant and the hemp plant. Cannabis is a genus of flowering plants in the family Cannabaceae. At least three species of this family are recognized: Cannabis sativa, Cannabis indica, and Cannabis ruderalis. Cannabis is also known as hemp, although this term is often used to refer only to varieties of Cannabis cultivated for non-drug use, i.e. having low or negligible contents of THC, for example, the principal psychoactive constituent.

However, the cannabinoids can also be obtained from other known sources of cannabinoids including other plants. Synthetic cannabinoids may also be employed. Various types of cannabinoids can be employed including those extracted in full spectrum, those extracted in broad spectrum, and cannabinoids in isolate form, all of which may be used either in a form that includes or excludes terpenes.

Exemplary cannabinoids include, but are not limited to, cannabidiol (CBD), tetra-hydrocannabinol (THC), cannabigerol (CBG), cannabinol (CBN), cannabichromene (CBC). Preferably, the entire plant extract is employed, rather than isolated active ingredients. Suitable extraction methods are known to skilled persons.

There are 483 identifiable chemical constituents known to exist in the cannabis plant, and at least 85 different cannabinoids have been isolated from the plant. The two cannabinoids usually produced in greatest abundance are cannabidiol (CBD) and/or A-tetrahydrocannabinol (THC), but only THC is psychoactive. Since the early 1970s, Cannabis plants have been categorized by their chemical phenotype or “chemotype”, based on the overall amount of THC produced, and on the ratio of THC to CBD. Although overall cannabinoid production is influenced by environmental factors, the THC/CBD ratio is genetically determined and remains fixed throughout the life of a plant.

Optionally, the composition of the present invention may also include hemp seed oil. Hemp seed oil typically contains essential amino acids, essential fatty acids and omega fatty acids for promoting cell regeneration.

In each of the foregoing embodiments, the composition may also comprise one or more of the following components: b2) DMSO/Dimethyl Sulfoxide, b3) minerals, b4) botanicals, b5) Vitamins, b6) Amino Acids, and b7) fungi.

Thus, another aspect of the present invention provides pharmaceutically acceptable compositions comprising a therapeutically effective amount of one or more of the compounds described above, formulated together with one or more pharmaceutically acceptable carriers (additives) and/or diluents. The oral dosage level can be 1-10,000 mg Ganoderma Lucidum spore oil per day, and 1-5,000 mg of the one or more cannabinoids per day. Preferably, the oral dosage level can be 10-4000 mg Ganoderma Lucidum spore oil per day, and 1-500 mg of the one or more cannabinoids per day. More preferably, the oral dosage level can be 90-3200 mg Ganoderma Lucidum spore oil per day, and 10-250 mg of the one or more cannabinoids per day.

The transdermal dosage level can be 1-200 mg Ganoderma Lucidum spore oil per day, and 1-100 mg of the one or more cannabinoids per day. Preferably, the transdermal dosage level can be 5-100 mg Ganoderma Lucidum spore oil per day, and 1-50 mg of the one or more cannabinoids per day. More preferably, the transdermal dosage level can be 10-75 mg Ganoderma Lucidum spore oil per day, and 1-20 mg of the one or more cannabinoids per day.

The suppository dosage level can be 1-1000 mg Ganoderma Lucidum spore oil per day, and 1-1000 mg of the one or more cannabinoids per day. Preferably, the suppository dosage level can be 25-500 mg Ganoderma Lucidum spore oil per day, and 30-300 mg of the one or more cannabinoids per day. More preferably, the suppository dosage level can be 75-200 mg Ganoderma Lucidum spore oil per day, and 50-200 mg of the one or more cannabinoids per day.

The specific amount used in a particular case depends on a number of factors including the reason for administration, e.g. the ailment or need of the subject mammal, the severity of the ailment or need, and other factors known to skilled persons.

The phrase “therapeutically effective amount” as used herein means that amount of a compound, material, or composition comprising a compound of the present invention which is effective for producing some measurable therapeutic effect which is improved when compared to a placebo at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Actual dosage levels of the active ingredients in the compositions of this invention may be varied so as to obtain an amount of the active ingredients which are effective to achieve the desired therapeutic response for a particular patient, composition, and mode of administration, without being toxic to the patient.

The selected dosage level will depend upon a variety of factors including the activity of the particular compound of the present invention employed, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

One having ordinary skill in the art can readily determine the effective amount of the composition required. For example, doses of the compositions of the invention could be employed at levels lower than that required in order to achieve the desired therapeutic effect and gradually increase the dosage until the desired effect is achieved.

In general, a suitable daily dose of a compound of the invention will be that amount of the composition which is the lowest dose effective to produce a therapeutic effect. Such an effective dose will generally depend upon the factors described above. If desired, the effective daily dose of the active compound may be administered as two, three, four, five, six or more sub-doses administered separately at appropriate intervals throughout the day, optionally, in unit dosage forms.

The composition can be configured to be administered by a mode selected from the group consisting of intravenous injection, intramuscular injection, subcutaneous injection, topical administration, transdermal patch, suppository and oral. The composition can be in powder form and can be orally ingested directly as a powder, or can be added into a drink. In addition, the oral composition can be prepared for sublingual administration.

General methodology on pharmaceutical dosage forms is found in Ansel et al., Pharmaceutical Dosage Forms and Drug Delivery Systems (Lippencott Williams & Wilkins, Baltimore Md. (1999)). The oral dosages can be encapsulated in a hard or soft gel capsule shell. The oral dosages can also be in tablets, boluses, powders, granules or pastes for application to the tongue.

When administration is by topical administration, the composition may be formulated as solutions, gels, ointments, creams, jellies, suspensions, spray applied to the skin and the like, as are well known in the art. A mode of topical administration includes mixing the composition in water and soaking at least a part of the body in the water for at least 10 seconds, or at least 1 minute, or at least 10 minutes, or up to 2 hours, or up to 1 hour, or up to 30 minutes, or 10 seconds to 2 hours, or 10 seconds to 1 hour, or 10 seconds to 30 minutes. In an embodiment, the patient soaks up to and including the neck in water in a bathtub, or equivalent thereof. In some embodiments, administration is by means of a transdermal patch. When administration is by suppository (e.g., rectal or vaginal), the compositions may contain conventional suppository bases.

The composition can include carriers and excipients (including but not limited to buffers, carbohydrates, mannitol, proteins, polypeptides or amino acids such as glycine, antioxidants, bacteriostats, chelating agents, suspending agents, thickening agents and/or preservatives), water, oils, saline solutions, aqueous dextrose and glycerol solutions, other pharmaceutically acceptable auxiliary substances, such as tonicity adjusting agents, wetting agents emulsifiers and lubricants, such as sodium lauryl sulfate and magnesium stearate, as well as coloring agents, release agents, coating agents, sweetening, flavoring and perfuming agents, and the like. Exemplary carriers may include one or more ingredients selected from, but not limited to, water, Hemp, Coffee, Green Tea, Black tea, Matcha, Chai, Cacao, Cloves, Baobab, Ashwaganda, Ginseng, Moringa, Brahmi/Bacopa Monieri, Hibiscus, He Shoo Woo, Pau D'Arco, Matcha, Irish Sea Moss, Cinnamon, Oregano, Neem, Nettles, Arnica, Calendula, Comfrey, Chamomile, Valerian Root, Bladderwrack, Burdock, Tamanu, turmeric, Horsetail, Saw palmetto, Ginger, Turmeric, Cacao, Rooibos, Fennel, Echinacea, Basil, Thyme, Lemongrass, Lemon balm, Passionflower, Guanabana, Tulsi, Fo-ti, Vanilla, Coconut, Hemp, Cedarwood, Jasmine, Imortelle, Lavender, Rose, Geranium, Eucalyptus, Peppermint, Spearmint, Rosemary, Ylang Ylang, Lemon, Citronella, Sage, Plumeria, Imortelle, Shisandra, Astraxanthan (Algae derived), Maca, Egyptian Blue Lotus, Iboga, Kratom, coconut, Almond, Sweet Almond, Olive, Jojoba, Argan, Avocado, Grape, Castor, Prickly Pear, Sea Buckthorn, Apricot, Blackseed, Evening Primrose, Rosehip, flax, Carrot, sunflower, Cocoa, Mango, Shea, Lucuma, Indian rhubarb root (Rheum palmatum), Sheep sorrel (Rumex acetosella), Slippery elm (Ulmus rubra) Dandelion, Apple (Apple Pectin), Pineapple (Bromelain Pineapple Enzyme), Spirulina, Chlorella, Milk thistle, Wormwood, Black walnut, Shilajit, and Pine. Other pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations that are suitable for delivery of the components of the present composition.

In solid dosage forms of the invention for oral administration (capsules, tablets, pills, dragees, powders, granules and the like), the active ingredients can be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredients therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils, glycerol, tetrahydrofuryl alcohol, polyethylene glycols and fatty acid esters of sorbitan, and mixtures thereof.

The addition of the active compounds of the invention to animal feed is preferably accomplished by preparing an appropriate feed premix containing the active compounds in an effective amount and incorporating the premix into the complete ration.

Alternatively, an intermediate concentrate or feed supplement containing the active ingredient can be blended into the feed. The way in which such feed premixes and complete rations can be prepared and administered are described in reference books (such as “Applied Animal Nutrition”, W.H. Freedman and CO., San Francisco, U.S.A., 1969 or “Livestock Feeds and Feeding” 0 and B books, Corvallis, Ore., U.S.A., 1977).

The method of the present invention can be used to reduce symptoms in patients suffering from, for example, for one or more of obesity, insomnia, depression, dementia, brain fog, vision loss, anxiety, obsessive compulsive disorder, cancer, lupus, nausea, rheumatoid arthritis, migraines, poly cystic ovarian syndrome, menstrual pain, menopause, diabetes, nerve damage, sciatica, Epstein Barr virus, hypothyroidism, post-traumatic stress disorder, herpes, shingles, tremors, hpv, pathogenic viruses, asthma, Crohn's disease, Parkinson's disease, diverticulitis, osteoporosis, carpal tunnel syndrome, allergies, high cholesterol, inflammation, eczema, psoriasis, acne, tooth decay, oral bacteria, hair loss, rosacea, dermatitis, shingles, fungal infection, memory loss, trichotillomania, dermatillomania, urinary tract infections, wound, cancer and the effects of cancer-related treatments such as cachexia and wasting, damage caused by concussion and related symptoms, and symptoms caused from breast implant illness.

The effects of the composition of the invention may help to reduce symptoms of various diseases and inflammation. The composition may help to enhance macrophage activity therefore helping to remove toxins and debris from the body.

The method of the present invention can be used to promote rapid healing of wounds in patients. A method which “promotes rapid healing of a wound” results in the wound healing more quickly as a result of the treatment than a similar wound heals in the absence of the treatment. “Promotion of wound healing” can also mean that the method helps to regulates the proliferation and/or growth of, inter alia, keratinocytes, or that the wound heals with less scarring, less wound contraction, less collagen deposition and more superficial surface area. In certain instances. “promotion of wound healing” can also mean that certain methods of wound healing have improved success rates, (e.g., the take rates of skin grafts) when used together with the method of the present invention.

The method of the present invention can be used to reduce the symptoms of psoriasis, acne, and rosacea in a patient presenting with all of psoriasis, acne, and rosacea. A method which “reduces symptoms of psoriasis, acne, and rosacea” results in the greater reduction of severity or duration of all three of these problems as a result of the treatment than a similar treatment with a placebo.

The method of the present invention can be used to help reduce pain, reduce inflammation and improve cell rejuvenation in a patient presenting with pain, inflammation and age-accumulated damaged cells. A method which “reduces pain and inflammation and improves cell rejuvenation” results in the greater reduction of pain, inflammation and age-accumulated damaged cells as a result of the treatment than a similar treatment with a placebo.

The method of the present invention can be used to reduce the symptoms of depression, anxiety and hypothyroidism in a patient presenting with depression, anxiety and hypothyroidism. A method which “reduces the symptoms of depression, anxiety and hypothyroidism” results in a greater reduction in depression, anxiety and hypothyroidism as a result of the treatment than a similar treatment with a placebo.

Psoriasis is a chronic skin disease affecting approximately about 2-4 percent of the population worldwide. While the pathogenesis of psoriasis has not yet been fully elucidated, significant evidence indicates that epidermal changes occur as a secondary response to cellular immune infiltrates in the skin. Psoriasis is characterized by discrete areas of skin inflammation with redness, thickening, intense scaling, and in some cases, itching. The disease has significant impact on the quality of life of affected individuals, both physically and psychologically. The composition of the invention can help to reduce the signs of psoriasis, i.e. erythema, plaque elevation, and scaling to a greater extent than similar treatment with a placebo.

Acne vulgaris (acne) is a chronic skin disease involving blockage and/or inflammation of pilosebaceous units (hair follicles and their accompanying sebaceous gland). It can present as noninflammatory lesions, inflammatory lesions, or a mixture of both, affecting mostly the face but also the back and chest. Moderate to severe acne is characterized as acne vulgaris with an Investigator's Global assessment (IGA) score of at least moderate (3) acne severity. As used herein “acne” includes all forms of acne. e.g., P. acnes, A. nodulocycstic, A. conglabata, A. fitlminans, pyoderma faciale, induced forms (e.g., chloracne or bromidacne and steroidacne, medication-induced acne rashes [e.g., observed with some cancer therapy]) and combinations of other diseases with acne, e.g., Synovitis-Acne-Pustulosis Hyperostosis-Osteitis (SAPHO), acne tetrad and follicular triad. As used herein “acne” also refers to non inflammatory and/or inflammatory acne. Disclosed herein is a method for preventing or reducing the symptoms of a patient having acne, e.g., moderate to severe acne, e.g. non-inflammatory acne, inflammatory acne or non-inflammatory and inflammatory acne.

Rosacea is a chronic inflammation that affects the wrinkles and face, especially the cheeks, chin, nose and forehead, of a middle-aged adult sexual skin condition. Common clinical symptoms are erythema (redness), marked angiogenesis, dryness, papules, pustules, swelling, vasodilation, lesions, inflammation, infection, enlarged nose, enlarged sebaceous glands, and mainly including nodules, alone or in combination, in the relevant skin area of the center of the face. Some of these clinical symptoms, especially erythema, are thought to result from vasodilation. In rare cases, rosacea may also occur on the torso and end, such as the chest, neck, back or scalp. Topical application of the composition can reduce redness, flushing, and blush associated with either rosacea or sensitive skin. After removal of rosacea, the topical composition can be administered once a day to encourage the skin to remain free of rosacea.

Inflammation is a process by which microbes or tissue injury induce the release of cytokines and chemokines from various cell types producing increased blood vessel permeability, upregulation of endothelial receptors, and thus increased egress of various cells of the innate and adaptive immune system which enter surrounding tissue and grossly produce the classical picture of inflammation, i.e. redness, swelling, heat and pain. Inflammation is a localized reaction of live tissue due to an injury, which may be caused by various endogenous and exogenous factors. The exogenous factors include physical, chemical, and biological factors. The endogenous factors include inflammatory mediators, antigens, and antibodies. Endogenous factors often develop under the influence of an exogenous damage. An inflammatory reaction is often followed by an altered structure and penetrability of the cellular membrane. Endogenous factors, namely, mediators, antigens, and autogens define the nature and type of an inflammatory reaction, especially its course in the zone of injury. In the case where tissue damage is limited to the creation of mediators, an acute form of inflammation develops. If immunologic reactions are also involved in the process, through the interaction of antigens, antibodies, and autoantigens, a long-term inflammatory process will develop. Various exogenous agents, for example, infection, injury, radiation, also provide the course of inflammatory process on a molecular level by damaging cellular membranes which initiate biochemical reactions. The inventive composition is more effective in ameliorating the pathological condition or reducing the symptoms of the inflammation when compared to a placebo.

The present invention provides a method to alleviate the symptoms of pain regardless of the cause of the pain. The general term “pain” reduced by the present method includes traumatic pain, neuropathic pain, organ pain, and pain associated with diseases. Traumatic pain includes pain resulting from injury, post-surgical pain and inflammatory pain. Neuropathic pain includes neuropathic and idiopathic pain syndromes, and pain associated with neuropathy such as diabetic neuropathy, causalgia, brachial plexus avulsion, occipital neuralgia, fibromyalgia, gout, and other forms of neuralgia. Organ pain includes ocular, corneal, bone, heart, skin/burn, visceral (kidney, gall bladder, etc.), joint, and muscle pain. Pain associated with diseases includes pain associated with cancer, AIDS, arthritis, herpes and migraine. The present invention helps to reduce pain of varying severity, i.e. mild, moderate and severe pain; acute and chronic pain. The present invention is effective in reducing joint pain, muscle pain, tendon pain, and burn pain. The inventive composition is more effective in ameliorating or reducing the pain when compared to a placebo.

Depression is a common psychological problem and refers to a mental state of low mood and aversion to activity. Various symptoms associated with depression include persistent anxious or sad feelings, feelings of helplessness, hopelessness, pessimism, and/or worthlessness, low energy, restlessness, irritability, fatigue, loss of interest in pleasurable activities or hobbies, excessive sleeping, overeating, appetite loss, insomnia, thoughts of suicide, and suicide attempts. The presence, severity, frequency and duration of the above-mentioned symptoms vary on a case to case basis. In some embodiments, a patient may have at least one, at least two, at least three, at least four, or at least five of these symptoms. The composition of the invention may help to relieve the patient of at least one symptom of depression for about 2 weeks or less, 1 week or less, 1 day or less, or 1 hour or less. (e.g. 15 minutes or less, half an hour or less), after administration. In some embodiments, such methods may help to relieve the patient of at least one symptom of depression for about 1 day or more, 1 week or more, or 2 weeks or more after administration.

The inventive composition can be used to help reduce anxiety. Examples of Anxiety Disorders include, but are not limited to, Panic Disorders, Agoraphobia, Specific Phobias, Social Phobias, Obsessive-Compulsive Disorder, Post-Traumatic Stress Disorder, Acute Stress Disorder, Generalized Anxiety Disorder, Substance-Induced Anxiety, Anxiety Related to Medical Disorders, Anxiety Disorder Not Otherwise Specified (NOS), as well as signs and symptoms of anxiety, stress, agitation, and worry that are not classified as an Anxiety Disorder. The composition of the invention may relieve the patient of anxiety for about 2 weeks or less, 1 week or less, 1 day or less, or 1 hour or less. (e.g. 15 minutes or less, half an hour or less, after administration. In some embodiments, such methods may help to relieve the patient of anxiety for about 1 day or more, 1 week or more, or 2 weeks or more after administration.

Thyroid underactivity (hypothyroidism) can be due to a dietary lack of iodine, the lack of which prevents thyroid cell synthesis of the thyroid hormones. In the Western world, Hashimoto's disease is the most common cause of hypothyroidism. Hypothyroidism can also result from radio-iodine treatment or surgery, to correct thyroid overactivity, as well as to a pituitary disorder. A method for reducing the symptoms of hypothyroidism according to the present invention can comprise the step of administration of a therapeutically effective amount of the composition, thereby increasing a deficient thyroid hormone secretion from the thyroid and effectively helping to reduce the symptoms of the hypothyroidism. The inventive composition is more effective in reducing symptoms of hypothyroidism when compared to a placebo.

The term “treatment” is intended to signify therapy for relief or reduction of symptoms.

The patient receiving this treatment is any animal in need, including primates, in particular humans, and other mammals such as equines, cattle, swine, dogs, cats, and sheep; and poultry and pets in general. It is preferred that the patient is a human.

The composition of the invention can be administered as such or in admixtures with pharmaceutically acceptable and/or sterile carriers and can also be administered in conjunction with other known active pharmaceutical compounds. Conjunctive therapy, thus includes sequential, simultaneous and separate administration of the active components in a way that the therapeutical effects of the first administered one is not entirely disappeared when the subsequent is administered.

### EXAMPLES

Example 1: A female human in her mid-50s struggling with diabetes, severe rheumatoid arthritis, obesity, insomnia, allergies, asthma and inflammation. This subject began taking 1 ingestible capsule a day. After day 1, the subject regained dexterity in her hands and reported that all pain had subsided. She continued ingesting 1 capsule per day and began combining this with topical cream application in low dosages to enhance the anti-inflammatory effects. Prior to initiation of the capsule regimen, she went from 6 hours of broken sleep per night while using a prescription sleep aid to 8-10 solid hours of deep REM sleep a night after only a few days of the capsule regiment. The subject also reported enhanced mental clarity and positive mood. After 1 week of use, the subject reports positive improvements from all ailments.

In this case, the daily dosage of the sublingual/ingestible supplements included: 16.5 mg of the full spectrum CBD+500 mg of Ganoderma Lucidum spore oil. The daily dosage of the transdermal was a microdose of 5 mg of the full spectrum CBD+50 mg Ganoderma Lucidum spore oil.

EXAMPLE 2: A human female in her mid-20s that previously suffered from severe polycystic ovarian syndrome, menstrual cramps, cystic acne, skin diseases, hormonal imbalances, anxiety and depression began taking 1 capsule in the morning, and 1 capsule in the evening. The subject reported that this dosage regimen enhanced her mental state and clarity, calmed stress previously causing depression, dermatillomania and trichotillomania, and provided relief from the pain and itching of eczema and psoriasis with visual improvement. Noted cosmetic improvements included healing of scar tissue and new hair follicle growth on her scalp, nail growth, skin health improvement including elimination of acne, scars faded and it provided an overall clear complexion. The subject also used a vaginal suppository and felt relief from cramping related to severe menstrual pain. Finally, the subject reported a significant improvement of quality of life.

In this case, the daily dosage of the sublingual/ingestible supplements included: 33 mg cannabinoids and 1000 mg Ganoderma Lucidum spore oil. For the transdermal application the daily dose was: 2 mg cannabinoids and 50 mg Ganoderma Lucidum spore oil. The suppository contained: 100 mg cannabinoids+100 mg Ganoderma Lucidum spore oil, and 1-5 suppositories were used per month, as needed.

EXAMPLE 3: A human female in her mid-20s with Lupus had previously scheduled a kidney transplant from initial stages of failure, extreme illness and discomfort. After 1 capsule, pain and nausea subsided for 24 hours, and insomnia was eliminated. The subject continued use of the composition and delivery through sublingual/ingestible, transdermal, and suppository delivery methods. The subject uses 1-3 suppositories a month and noted feeling clean and refreshed with immediate relief of menstrual cramping. Cosmetic improvements were also observed in health and quality of skin and hair. Subject is pain free and reported a significant improvement in quality of life.

The daily dosage of the sublingual/ingestible supplements was: 200 mg cannabinoids and 3000 mg Ganoderma Lucidum spore oil. For the transdermal application: 10 mg of cannabinoids and 50 mg of Ganoderma Lucidum spore oil was used. The suppository contained: 75 mg of cannabinoids and 100 mg of Ganoderma Lucidum spore oil. 1-3 suppositories per month were used as needed.

EXAMPLE 4: A human male in his early 60s suffering from severe pain from inflammation, previous injury and loss of cartilage in knees and ankles resulting in the need for replacement, back pain, numbness in limbs, restless leg syndrome, sleep apnea, insomnia, and mental issues of manic depression, anxiety and bipolar disorder. The subject began using topical cream on all areas of severe pain and felt relief within 15 minutes, lasting 24 hours. After one week, subject introduced 2 daily ingestible capsules into the regimen taken both morning and evening, and noted an immediate sense of calmness and well-being with both physical and physiological improvements. Depression, insomnia, sleep apnea, inflammation and pain has significantly subsided. Cosmetic improvements were also observed in health and quality of skin and hair. The subject reported a significant improvement on quality of life.

The daily dosage of the sublingual/ingestible supplements was: 33 mg cannabinoids and 1000 mg Ganoderma Lucidum spore oil. For the transdermal application: 6 mg of cannabinoids and 16.5 mg of Ganoderma Lucidum spore oil was used.

EXAMPLE 5: A human male in his mid-70s suffers from extremely rare non Hodgkins Burkitts cancer, melanoma, and prostate cancer. The subject took 1 ingestible capsule and felt relief from all pain in 15 minutes which consistently lasted 24 hours. The subject began taking capsules twice a day every day and incorporating topical cream into the regimen. The subject reported an increase in energy, was pain free, experienced solid night's sleep and stated: “I haven't felt this great in over twenty years since before I got cancer.” The subject also reported a significant improvement in quality of life.

The daily dosage of the sublingual/ingestible supplements was: 16.5 mg of full spectrum cannabinoids, 500 mg Ganoderma Lucidum spore oil and hemp seed oil. For the transdermal application: 5 mg of full spectrum cannabinoids and 50 mg of Ganoderma Lucidum spore oil was used.

EXAMPLE 6: A human female in her 50s battling lung cancer for ten years and bed ridden from pain and nausea consumed 2 capsules and was able to take a 6 hour car ride without pain or nausea. The subject now takes 4 capsules daily, 2 in the morning/2 in the evening. The subject reported an overall improvement in health without pain, and relief from the side effects associated with the treatment of cancer. The subject also reported a significant improvement in quality of life.

The daily dosage of the sublingual/ingestible supplements was: 200 mg cannabinoids and 3000 mg Ganoderma Lucidum spore oil. For the transdermal application: 10 mg of cannabinoids and 50 mg of Ganoderma Lucidum spore oil was used.

EXAMPLE 7: A human female in her late 80s suffered from severe shingles. The subject applied topical cream to eliminate itching sensation and clear wounds. The subject reported that she felt relief within minutes of application. The subject subsequently began taking ingestible supplements and noticed relief, increased energy, and an improvement in overall well-being. The subject reported a significant improvement in quality of life.

The daily dosage of the sublingual/ingestible supplements was: 16.5 mg cannabinoids and 500 mg Ganoderma Lucidum spore oil. For the transdermal application: 1 mg of cannabinoids and 15 mg of Ganoderma Lucidum spore oil was used.

EXAMPLE 8: A human female in her early 30s suffering from post-traumatic stress disorder, anxiety, migraines, and carpal tunnel applied topical cream for transdermal delivery and felt relief within fifteen minutes lasting 24 hours. Cosmetic improvements were also observed in health and quality of skin, hair, and nails.

The daily dosage of the transdermal application was: 5 mg of cannabinoids and 30 mg of Ganoderma Lucidum spore oil was used.

EXAMPLE 9: A human female in her late 20s suffered from depression, anxiety, trichotillomania, obsessive compulsive disorder, insomnia, dermatillomania, cystic acne and welts on her entire body, hormonal imbalance, severe menstrual cramping, cortisol level issues, chronic ear infections, chronic urinary tract infections, polycystic ovarian syndrome, tooth decay related to deficiencies and virus, memory loss, fatigue, and symptoms caused by breast implant illness including numbness and pain in limbs. This subject began taking sublingual/ingestible capsules daily in combination with daily transdermal delivery, and suppository use once a month. She noted relief of pain and discomfort within minutes, and increased improvement with use. After two weeks of use, all cosmetic issues subsided with improvements observed in overall health and quality of skin, hair, and nails. The subject also noted more energy, vision improvement, oral decay improvement, mental clarity and memory recall, mood stability, painlessness, and a significant improvement in quality of life.

The daily dosage of the sublingual/ingestible supplements was: 130 mg cannabinoids and 2000 mg Ganoderma Lucidum spore oil. For the transdermal application: 10 mg of cannabinoids and 50 mg of Ganoderma Lucidum spore oil was used. The suppository contained: 100 mg of cannabinoids and 100 mg of Ganoderma Lucidum spore oil. 1-5 suppositories per month were used as needed.

EXAMPLE 10: A human female in her late 50s suffering from herpes, sciatica, five herniated discs, migraines, tremors, memory loss and brain fog, fatigue, anxiety, menopause and related symptoms, breast implant illness, and arthritis and who previously suffered from insomnia average sleep 3-5 hours daily took 1 sublingual/ingestible capsule per day and slept 8 solid hours a night without hot flashes, menopause related symptoms, or pain. Memory recall, noted memories from decades ago with acutely detailed recall. The subject began ingesting supplements and using topical cream daily, and suppositories, when necessary. The subject reported that pain, insomnia, and symptoms subsided and noted a significant improvement in quality of life from the combination of delivery methods. The subject also noted complete memory recall in acute detail, increased energy, and mental clarity. Cosmetic improvements were also observed in health and quality of skin, hair, and nails.

The daily dosage of the sublingual/ingestible supplements was: 33 mg cannabinoids and 1000 mg Ganoderma Lucidum spore oil. For the transdermal application: 2 mg of cannabinoids and 50 mg of Ganoderma Lucidum spore oil was used. The suppository contained: 100 mg of cannabinoids and 100 mg of Ganoderma Lucidum spore oil. 1-5 suppositories per month were used as needed.

EXAMPLE 11: A human female in her early 20s suffered from numerous near-fatal concussions causing severe discomfort lasting years, brain damage and inflammation leading to full body discomfort, constant pain in head, neck, and back. These incidents lead to tremors, memory loss, sensitivity to light, brain fog, anxiety, fatigue, restlessness. The subject also suffered from consistent urinary tract infections. The subject also experienced pregnancy complications leading to miscarriage and surgical removal with extreme pain and discomfort. The subject began taking sublingual/ingestible supplements daily, coupled with topical application for transdermal delivery and insertion of vaginal suppository for relief.

The subject noted a significant improvement in quality of life from the combination of delivery methods. Brain injury symptoms subsided and pain was significantly reduced. Mental clarity returned and insomnia was eliminated. Cosmetic improvements were also observed in health and quality of skin, hair, and nails.

The daily dosage of the sublingual/ingestible supplements was: 100 mg cannabinoids and 1500 mg Ganoderma Lucidum spore oil. For the transdermal application: 2 mg of cannabinoids and 50 mg of Ganoderma Lucidum spore oil was used. The suppository contained: 100 mg of cannabinoids and 100 mg of Ganoderma Lucidum spore oil. 1-5 suppositories per month were used as needed.

### B1) Cannabinoid

In each of the foregoing embodiments, the composition can include a combination of Mushroom Spore Oil and/or Spore Powder and at least one cannabinoid. Cannabis has naturally occurring terpenes which offer an “entourage effect” and further enhance the effects of cannabinoids.

Terpenes include but are not limited to: Limonene, Caryophyllene, Humulene, Pinene, Myrcene, Bisabolol, Carene, Terpinene, Borneol, Camphene, Eucalyptol, Geraniol, Phytol, Terpinolene, Nerolidol, and Valencene.

I have discovered that there is a further interaction and maximization between the terpenes of the cannabis plant and the tri-terpenes in the mushroom. This new composition has been found to maximize results as a method for enhancing/stimulating the immune, endocannabinoid and/or main bodily systems of a patient, and reducing symptoms, particularly in a mammal with a disease or disorder.

The composition comprising cannabinoids can be, but is not limited to topical, oral, and suppository formulations.

This composition may also be combined with b2) Dimethyl Sulfoxide (DMSO) b3) Minerals, b4) botanicals, b5) Vitamins, b6) Amino Acids, and b7) fungi.

### B2) Dimethyl Sulfoxide (DMSO)

In each of the foregoing embodiments, the composition can include a combination of Mushroom Spore Oil and/or Spore Powder and Dimethyl Sulfoxide (DMSO).

The composition comprising DMSO can be, but is not limited to topical, oral, and suppository formulations.

This composition may also be combined with one or more selected from b1) a cannabinoid, b3) minerals, b4) botanicals, b5) Vitamins, b6) Amino Acids, and b7) fungi.

### B3) Mineral Compositions

In each of the foregoing embodiments, the composition can include a combination of Mushroom Spore Oil and/or Spore Powder and at least one of the following minerals in pharmaceutically acceptable form: magnesium such as magnesium sulfate, potassium, silica, sodium, zinc, boron, chloride, iron, Himalayan salt, fluoride, and Shilajit.

The composition comprising mineral compositions can be, but is not limited to topical, oral, and suppository formulations.

This composition may also be combined with one or more of b1) a cannabinoid, b2) Dimethyl sulfoxide (DMSO), b4) botanicals, b5) Vitamins, b6) Amino Acids, and b7) fungi.

The following example includes the topical administration of a composition comprising a b3) mineral composition.

EXAMPLE 12: A woman in late 20s was experiencing shin splits, sciatica tight muscles, aching back pains, and circulation issues after severe car accident which also resulted in a concussion. The woman tried soaking body in 1 cup of Magnesium sulfate with 20 gallons of hot water for 30 minutes. The woman noticed a slight improvement with one use. After 72 hours, the woman soaked her body in 20 gallons of hot water comprising 1 cup of Magnesium sulfate in combination with 1000 mg of Reishi Mushroom Spore Powder, for 30 minutes. The woman noticed an extreme improvement in her overall pain relief, better sleep, improved mood, circulation, and dexterity that lasted into the next day.

### B4) Botanical Compositions

In each of the foregoing embodiments, the composition can include a combination of Mushroom Spore Oil and/or Spore Powder and at least one botanical compositions. The composition may also include Active Plant Stem Cells, wherein the active plant stem cells may be in powder or liquid form, and are combined with mushroom spore oil and/or powder.

The composition may also include plant terpenes. Terpenes include but are not limited to: Limonene, Caryophyllene, Humulene, Pinene, Myrcene, Bisabolol, Carene, Terpinene, Borneol, Camphene, Eucalyptol, Geraniol, Phytol, Terpinolene, Nerolidol, and Valencene.

The composition comprising botanical compositions can be, but is not limited to topical, oral, and suppository formulations.

The composition may also be combined with one or more b1) a cannabinoid, b2) Dimethyl sulfoxide (DMSO), b3) minerals, b5) Vitamins, b6) Amino Acids, and b7) fungi.

Botanical plants, herbs, kelps, algae, and their leaves, bark, flowers, seeds, roots, fruits and/or beans, can be used in dry powder form, can be utilized/extracted into liquid form and/or into carrier liquids, oils and/or powders. The botanicals can be brewed and consumed as tea and/or coffee. The composition comprising botanicals can also be prepared into consumable food goods/superfoods. The composition can comprise botanicals which optionally can be extracted into essential oil form.

Botanicals for use in the composition include one or more of the following:
1. Hemp,
2. Coffee,
3. Green Tea,
4. Black tea,
5. Matcha,
6. Chai,
7. Cacao,
8. Cloves,
9. Baobab
10. Ashwaganda,
11. Ginseng,
12. Moringa,
13. Brahmi/Bacopa Monieri,
14. Hibiscus,
15. He Shoo Woo,
16. Pau D'Arco,
17. Matcha,
18. Irish Sea Moss,
19. Cinnamon,
20. Oregano,
21. Neem,
22. Nettles,
23. Arnica,
24. Calendula,
25. Comfrey,
26. Chamomile,
27. Valerian Root,
28. Bladderwrack,
29. Burdock,
30. Tamanu,
31. turmeric,
32. Horsetail,
33. Saw palmetto,
34. Ginger,
35. Turmeric,
36. Cacao,
37. Rooibos,
38. Fennel,
39. Echinacea,
40. Basil,
41. Thyme,
42. Lemongrass,
43. Lemon balm,
44. Passionflower,
45. Guanabana,
46. Tulsi,
47. Fo-ti,
48. Vanilla,
49. Coconut,
50. Cedarwood,
51. Jasmine,
52. Imortelle,
53. Lavender,
54. Rose,
55. Geranium,
56. Eucalyptus,
57. Peppermint,
58. Spearmint,
59. Rosemary,
60. Ylang Ylang,
61. Lemon,
62. Citronella,
63. Sage,
64. Plumeria,
65. Imortelle,
66. Shisandra,
67. Astraxanthan (Algea derived),
68. Maca,
69. Egyptian Blue Lotus,
70. Iboga,
71. Kratom,
72. coconut,
73. Almond,
74. Sweet Almond,
75. Olive,
76. Jojoba,
77. Argan,
78. Avocado,
79. Grape/Grapeseed,
80. Castor,
81. Prickly Pear,
82. Sea Buckthorn,
83. Apricot,
84. blackseed,
85. Evening primrose,
86. Rosehip,
87. Flax,
88. Carrot,
89. Sunflower,
90. Cocoa,
91. Mango,
92. Shea (Vitellaria paradoxa),
93. Lucuma,
94. Indian rhubarb root (Rheum palmatum),
95. Sheep sorrel (Rumex acetosella),
96. Slippery elm (Ulmus rubra),
97. Dandelion,
98. Apple (Apple Pectin),
99. Pineapple (Bromelain Pineapple Enzyme),
100. Spirulina,
101. Chlorella,
102. Milk thistle,
103. Wormwood,
104. Black walnut
105. Shilajit
106. Pine.

The composition may be sustained in a carrier. Exemplary carriers may include one or more ingredients selected from, but not limited to the botanical ingredients listed above as well as any other pharmaceutically acceptable carrier known to persons skilled in the art that is suitable for delivery of the components of the composition.

The composition comprising botanicals can be, but is not limited to topical, oral, and suppository formulations.

### Coffee and Tea Compositions

Coffee is one of the above-listed botanicals that can be used in the composition. Coffee is a popular beverage that researchers have studied extensively for its many health benefits. It contains several antioxidants, including hydrocinnamic acids and polyphenols, which may play a role in preventing several chronic and acute conditions. Some of the health benefits of coffee include increasing energy levels, promoting weight management, enhancing athletic performance, and protecting against chronic disease.

My invention includes a composition that comprises: a) mushroom spore oil and/or powder; b) coffee and/or tea; and/or c) a cannabinoid and the benefits derived therefrom. In other words, the composition can include any one of the following open-ended sets: (a, b), (a, b, c), and (a, c).

The coffee beans can be roasted. The coffee beans can be ground and/or brewed, and consumed orally and/or as a beverage. This coffee can also be administered as a suppository or enema. It can also be administered topically. Ground Coffee applied topically has additional benefits for the skin, making the skin appear tighter, more youthful, evening the tone and texture. The composition with specified mushroom spore oil and/or powder and coffee may also include at least one of b1) a cannabinoid, b2) DMSO/Dimethyl Sulfoxide, b3) minerals, b4) other botanicals, b5) Vitamins, b6) Amino Acids, and b7) fungi; especially preferred is b4) other botanicals for enhanced synergistic effects.

The composition comprising coffee can be, but is not limited to topical, oral, and suppository formulations.

The following examples include the topical and oral administration of a composition comprising coffee.

EXAMPLE 13: A woman in her late 20's struggling with depression, drank chamomile tea to aid with these issues. She noticed very little difference. She took 500 mg of Reishi mushroom spore and noticed some improvement. The next day she consumed a combination of chamomile tea and 500 mg of reishi mushroom spore and noticed greater improvements in her mood, sleep, and relaxation. For even greater levels of enhancement, she then consumed a combination of chamomile tea with 500 mg of reishi mushroom spore and 15 mg of Full Spectrum CBD. The results proved that the synergistic effects of the mushroom spore with chamomile were greater combined than the individual ingredient results. The effects were even greater when the 3rd component of a cannabinoid were included.

Example 14: A man in his early 20s with severe acne, tried many individual ingredients on separate days. The ingredients included sweet almond oil, organ oil, hemp seed oil, rosehip seed oil, and he only saw a minor improvement. After 7 days, the man applied hemp CBD oil to his skin to test the results, and he noticed a minor overall improvements. After 7 more days he applied mushroom spore oil directly to his skin, and again he noticed minor improvements. The following week, he applied Hemp CBD and Mushroom spore oil to his skin and noticed profound changes and overall improvement to the tone and texture of his skin.

EXAMPLE 15: A woman in her early 20s suffering from rosacea, eczema, psoriasis, stretch marks and acne, applied a composition including ground roasted coffee, 500 mg mushroom spore oil, and 15 mg full spectrum CBD to her skin, rubbing in a circular motion to the affected area for five minutes. After one week, the woman noticed an improved texture and tone of skin, reduced redness from rosacea, flaking and itch from eczema and psoriasis subsided completely. The overall appearance of her skin improved drastically.

EXAMPLE 16: A man in his mid 50's who would drink one cup of coffee every day, suffered from brain fog, indigestion, jitters, anxiety, depression, sleeplessness, and lack of focus. He previously tried taking herbs, including ashwaganda, but did not notice much benefit. He consumed 1 cup of coffee with 500 mg mushroom spore powder and noticed a sense of overall calm, mental clarity, focus, attention to detail, and overall health. The following week, he consumed a composition with coffee, 500 mg mushroom spore, and ¼ tsp of Ashwaganda. The man noticed an even greater result and no longer felt that he suffered from brain fog, indigestion, jitters, anxiety, depression, sleeplessness, lack of focus.

### B5) Vitamin Compositions

In each of the foregoing embodiments, the composition can include a combination of Mushroom Spore Oil and/or Spore Powder and at least one of the following vitamins: Vitamin E, Vitamin C, Vitamin D, and Vitamin K. These vitamins may be in powder or liquid form.

The composition comprising vitamins can be, but is not limited to topical, oral, and suppository formulations.

This composition may also be combined with one or more of b1) a cannabinoid, b2) Dimethyl sulfoxide (DMSO), b3) minerals, b4) botanicals, b6) Amino Acids, and b7) fungi.

### B6) Amino Acid Compositions

In each of the foregoing embodiments, the composition can include a combination of Mushroom Spore Oil and/or Spore Powder and at least one amino acid selected from Leucine, Valine, Isoleucine, Phenylalanine, Lysine, Histidine, Methionine, Tryptophan, and Threonine.

The composition can be, but is not limited to topical, oral, and suppository formulations.

This composition may also be combined with one or more of b1) a cannabinoid, b2) Dimethyl sulfoxide (DMSO), b3) minerals, b4) botanicals, b5) Vitamins, and b7) fungi.

### B7) Fungi Compositions

In each of the foregoing embodiments, the composition can include a combination of Mushroom Spore Oil and/or Spore Powder with the mushroom fruiting body and/or mycelium of one or more of:
i. Reishi (Ganoderma lucidum),
ii. Lion's Mane (Hericium Erinaceus),
iii. Cordyceps (Cordyceps Militaris),
iv. Turkey Tail (Trametes versicolor/Coriolus versicolor),
v. Agaricus (Agaricus Blazei),
vi. Chaga (Inonotus Obliquus),
vii. Tremella (Tremella fuciformis),
viii. Shiitake (Lentinula edodes),
ix. Maitake (Grifola frondosa),
x. Oyster (Pleurotus ostreatus),
xi. Indian Oyster (Pleurotus Pulmonarius),
xii. Poria (Wolfiporia cocos),
xiii. Boletus (Boletus edulis),
xiv. Morel (Morchella esculenta),
xv. Amanita (Amanita Muscaria),
xvi. Trumpet (Pleurotus eryngii),
xvii. Psilocybe,
xviii. Usnea Lichen,
xix. Lobaria pumponaria Lichen,
xx. Cetraria Islandica/Icelandic Moss Lichen, and
xxi. Evernia Lichen.

The composition can be, but is not limited to topical, oral, and suppository formulations.

The composition may also comprise one or more of the following components:
b1) a cannabinoid, b2) Dimethyl sulfoxide (DMSO), b3) minerals, b4) botanicals, b5) Vitamins, and b6) Amino Acids.

The following example includes the oral administration of a composition comprising the Lion's mane mushroom fruiting body.

EXAMPLE 17: A woman in her 30's was suffering from brain fog, upset stomach, and lack of energy. She consumed Reishi spore powder capsules and noticed some improvement. Days after, she separately took Lion's mane fruiting body capsules and felt some improvement. The next week she took a capsule comprising the combination of Reishi Spore Powder with Lion's Mane fruiting body powder and noticed immense improvement and synergistic results that exceeded the individual results.

1. A composition comprising: a) a mushroom spore oil and/or powder; and at least one of b1) a cannabinoid, b2) DMSO/Dimethyl Sulfoxide, b3) minerals, b4) botanicals, b5) Vitamins, b6) Amino Acids, and b7) fungi;
wherein the mushroom spore oil and/or powder is based on at least one of the following mushrooms:
i. Reishi (Ganoderma lucidum)
ii. Lion's Mane (Hericium Erinaceus),
iii. Cordyceps (Cordyceps Militaris),
iv. Turkey Tail (Trametes versicolor/Coriolus versicolor),
v. Agaricus (Agaricus Blazei),
vi. Chaga (Inonotus Obliquus),
vii. Tremella (Tremella fuciformis),
viii. Shiitake (Lentinula edodes),
ix. Maitake (Grifola frondosa),
x. Oyster (Pleurotus ostreatus),
xi. Indian Oyster (Pleurotus Pulmonarius)
xii. Poria (Wolfiporia cocos),
xiii. Boletus (Boletus edulis),
xiv. Morel (Morchella esculenta),
xv. Amanita (Amanita Muscaria),
xvi. Trumpet (Pleurotus eryngii),
xvii. Psilocybe,
xviii. Usnea Lichen,
xix. Lobaria pumponaria Lichen,
xx. Cetraria Islandica/Icelandic Moss Lichen, and
xxi. Evernia Uchen.

2. The composition according to claim 1, comprising both coffee and/or specified botanicals and a cannabinoid.

3. The composition according to claim 1, comprising Reishi (Ganoderma lucidum) spore oil and/or powder.

4. The composition according to claim 1, comprising a mushroom fruit body and/or mycelium extract of at least one of components i)-xxi).

5. The composition according to claim 1, further comprising b1) a cannabinoid.

6. The composition according to claim 1, wherein the cannabinoid is including but not limited to a full spectrum cannabinoid, broad spectrum cannabinoid, isolated cannabinoid, cannabidiol (CBD), Hemp CBD, tetra-hydrocannabinol (THC), cannabigerol (CBG), cannabinol (CBN), cannabichromene (CBC) or mixtures thereof.

7. The composition according to claim 1, comprising cannabis, or at least one terpene, or DMSO, or at least one mineral, or at least one botanical, or at least one vitamin, or at least one amino acid.

8. The composition according to claim 1, comprising coffee.

9. The composition according to claim 1, comprising tea and cannabinoid.

10. The composition according to claim 1, comprising Reishi Spore Powder and/or oil and Lion's Mane fruiting body powder.

11. The composition according to claim 1, which is formulated to be administered by a mode selected from the group consisting of intravenous injection, intramuscular injection, subcutaneous injection, topical administration, transdermal patch, suppository and oral.

12. A method of upregulating the immune system comprising administering a therapeutically effective amount of the composition according to claim 1, to a patient in need thereof.

13. A method of upregulating the endocannabinoid system comprising administering a therapeutically effective amount of the composition according to claim 1, to a patient in need thereof.

14. A method of upregulating the main bodily systems comprising administering a therapeutically effective amount of the composition according to claim 1, to a patient in need thereof.

15. A method of improving mental stability comprising administering a therapeutically effective amount of the composition according to claim 1, to a patient in need thereof.

16. A method of reducing symptoms of at least one of obesity, insomnia, depression, dementia, brain fog, vision loss, anxiety, obsessive compulsive disorder, cancer, lupus, nausea, rheumatoid arthritis, migraines, poly cystic ovarian syndrome, menstrual pain, menopause, diabetes, nerve damage, sciatica, Epstein Barr virus, hypothyroidism, post-traumatic stress disorder, herpes, shingles, tremors, hpv, pathogenic viruses, asthma, Crohn's disease, Parkinson's disease, diverticulitis, osteoporosis, carpal tunnel syndrome, allergies, high cholesterol, inflammation, eczema, psoriasis, acne, tooth decay, oral bacteria, hair loss, rosacea, dermatitis, shingles, fungal infection, memory loss, trichotillomania, dermatillomania, urinary tract infections, wound, cancer and the effects of cancer-related treatments such as cachexia and wasting, damage caused by concussion and related symptoms, and symptoms caused from breast implant illness comprising administering a therapeutically effective amount of the composition according to claim 1, to a patient in need thereof.

17. A method of reducing symptoms of acne, comprising administering the composition according to claim 1, to a patient in need thereof.

18. A method of reducing symptoms of at least two of rosacea, eczema, psoriasis, stretch marks and acne, comprising administering the composition according to claim 1, to a patient in need thereof.

19. A method of reducing symptoms of at least two of brain fog, indigestion, jitters, anxiety, depression, and sleeplessness, comprising administering the composition according to claim 1, to a patient in need thereof.

20. A method of reducing symptoms of at least two of brain fog, upset stomach, and lack of energy, comprising administering the composition according to claim 1, to a patient in need thereof.

21. A composition comprising: a) a mushroom spore oil and/or powder; and at least one of b1) DMSO/Dimethyl Sulfoxide, b2) minerals, b3) botanicals, b4) Vitamins, b5) Amino Acids, and b6) fungi;
wherein the mushroom spore oil and/or powder is based on at least one of the following mushrooms:
i. Reishi (Ganoderma lucidum)
ii. Lion's Mane (Hericium Erinaceus),
iii. Cordyceps (Cordyceps Militaris),
iv. Turkey Tail (Trametes versicolor/Coriolus versicolor),
v. Agaricus (Agaricus Blazei),
vi. Chaga (Inonotus Obliquus),
vii. Tremella (Tremella fuciformis),
viii. Shiitake (Lentinula edodes),
ix. Maitake (Grifola frondosa),
x. Oyster (Pleurotus ostreatus),
xi. Indian Oyster (Pleurotus Pulmonarius)
xii. Poria (Wolfiporia cocos),
xiii. Boletus (Boletus edulis),
xiv. Morel (Morchella esculenta),
xv. Amanita (Amanita Muscaria),
xvi. Trumpet (Pleurotus eryngii),
xvii. Psilocybe,
xviii. Usnea Lichen,
xix. Lobaria pumponaria Lichen,
xx. Cetraria Islandica/Icelandic Moss Lichen, and
xxii. Evernia Lichen.
